(19) United States
(12) Patent Application Publication
(10) Pub. No.: US 2005/0240196 A1
(43) Pub. Date: Oct. 27, 2005

(54) Apparatus for use in orthopaedic surgery

(76) Inventors: Kenneth P. Davis, High Wycombe (GB); David L. Mollon, Crowthorne (GB); Timothy S. Drew, Errol (GB); David W. Paterson, Darvil (GB); David I. Rowley, Kelly Castle (GB); Roy Smith, Lymington (GB)

Correspondence Address: Phillip L. Kenner;RENNER, KENNER, GREIVE,, BOBAK, TAYLOR & WEBER, First National Tower, Fourth Floor, Akron, OH, 44308-1456, US

(21) Appl. No.: 11/075,904

(22) Filed: Mar. 9, 2005

(30) Foreign Application Priority Data
Mar. 9, 2004 (GB) GB0405294.0
May 17, 2004 (GB) GB0410978.1
May 17, 2004 (GB) GB0410965.8

(51) Int. Cl. A61B 17/58

(52) U.S. Cl. 606/87

(57) ABSTRACT

Apparatus for use in orthopaedic surgery includes disposable or partially disposable cutting jigs for use on restructuring the femur and tibia as well as equipment for use in reshaping the patella, thus providing a complete kit for use in knee surgery.

The present invention relates to apparatus for use in orthopaedic surgery and more particularly to apparatus for use in preparing bones for the fitting of artificial joints.

It has become well known to replace worn or damaged human joints by artificial joints and one such joint is the knee. Successful total knee arthroplasty is directly dependent on re-establishment of normal lower extremity alignment, proper orientation of the artificial joint and secure fixation. Instruments and techniques to assist the surgeon in achieving the above factors already exist but are currently made of metal so that they can be sterilised by autoclaving after use. Unfortunately, autoclaving does not necessarily result in the instruments being automatically suitable for reuse and there exists a need for a single-use alternative to the current instruments.

It is an object of the present invention to provide disposable cutting jigs for use in orthopaedic surgery.

In one embodiment, the jig is in the form of a single unitary block which provides guides for all the necessary cuts to one of the bones. Alternatively, two jigs may be provided. In this case, the first jig provides for an initial cut while the second jig provides for all the remaining cuts.

We have also designed instruments specifically for the femur and tibia respectively so that preferably at least certain parts can be single use.

The knee joint also includes the patella and although it can be replaced, more often than not it is simply resurfaced.

A problem when carrying out resurfacing of the patella is that it is difficult to grip and maintain in a fixed position so as to be able to locate a drill guide onto it.

We have also designed instruments for operating on the patella.

In order that the present invention be more readily understood, embodiments thereof will now be described by way of example with reference to the accompanying drawings in which:

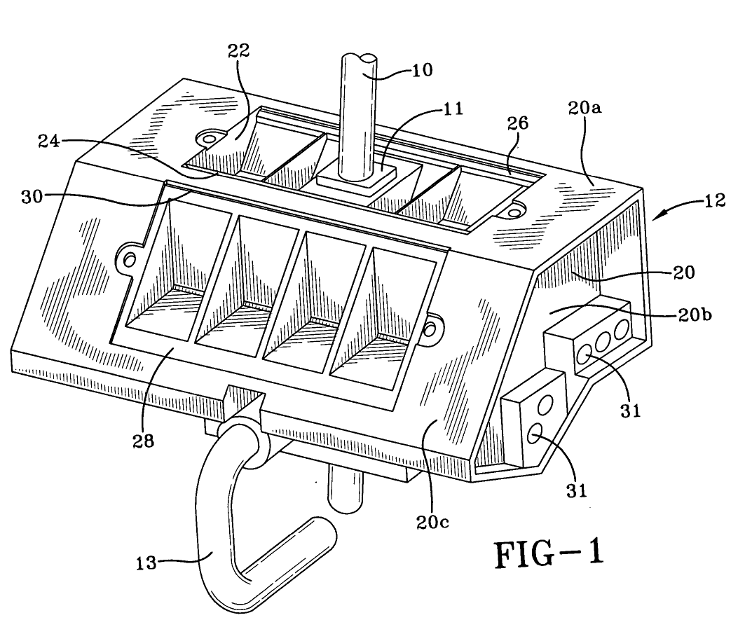

FIG. 1 shows a perspective view of an assembled kit of parts for a disposable cutting jig;

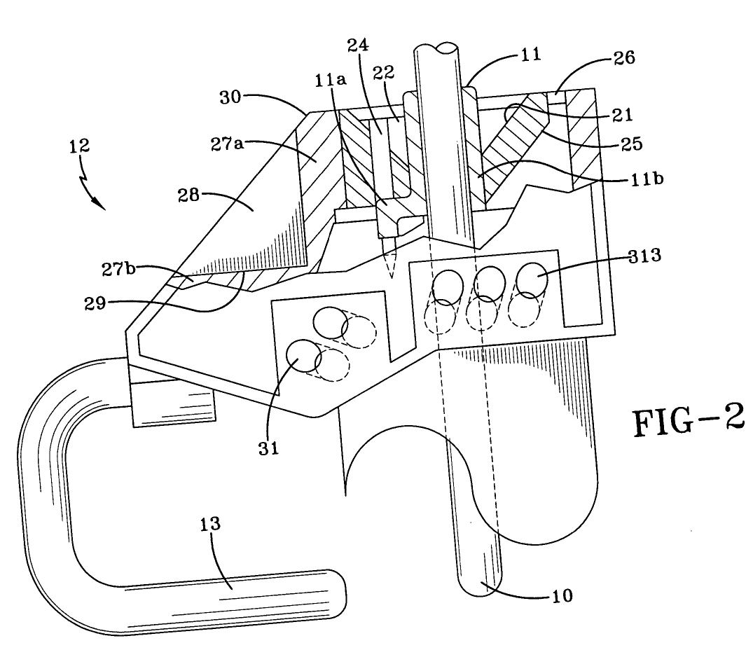

FIG. 2 is a diagrammatic cross-sectional view of the jig shown in FIG. 1 in position on a femur;

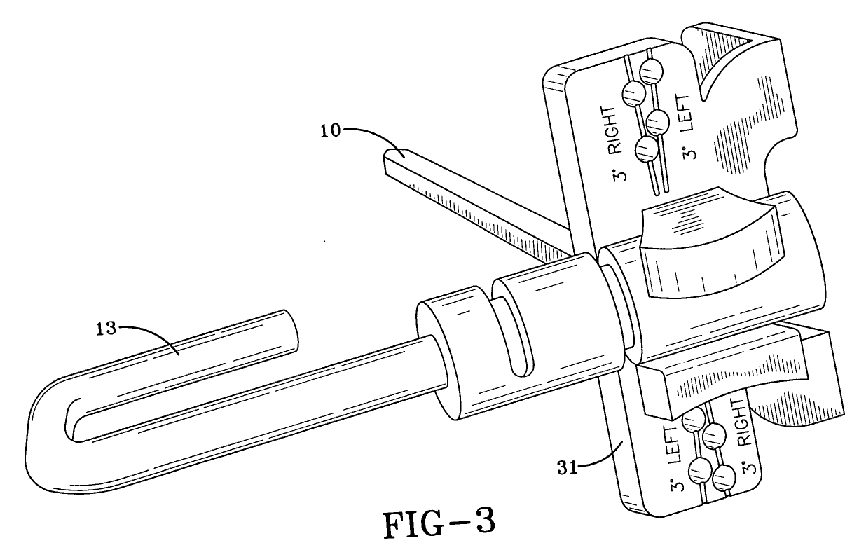

FIG. 3 is a perspective view of a part of the kit used to locate guide pins in position;

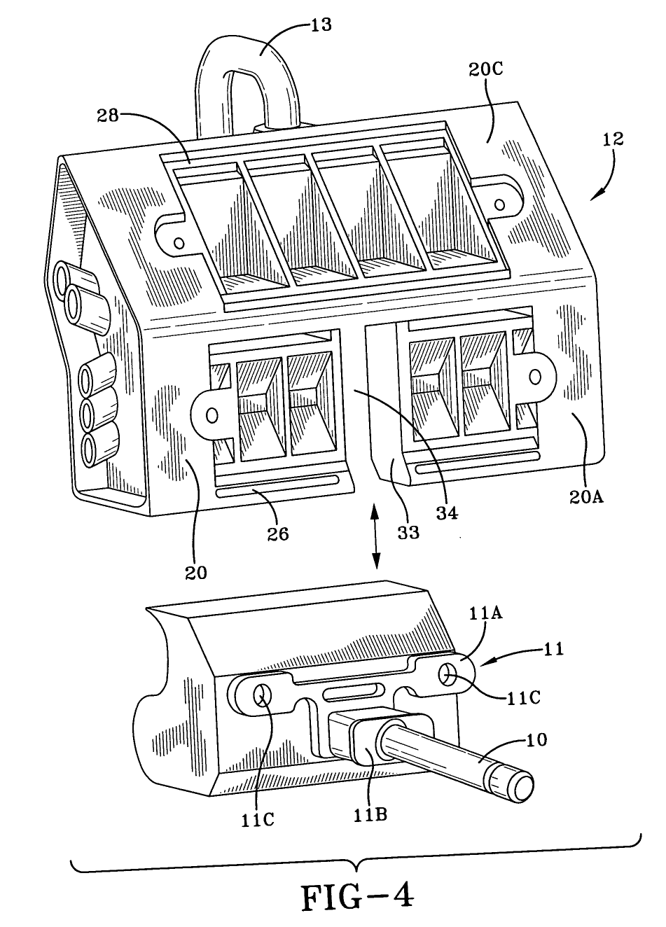

FIG. 4 shows a perspective view of a modification to the embodiment shown in FIG. 1;

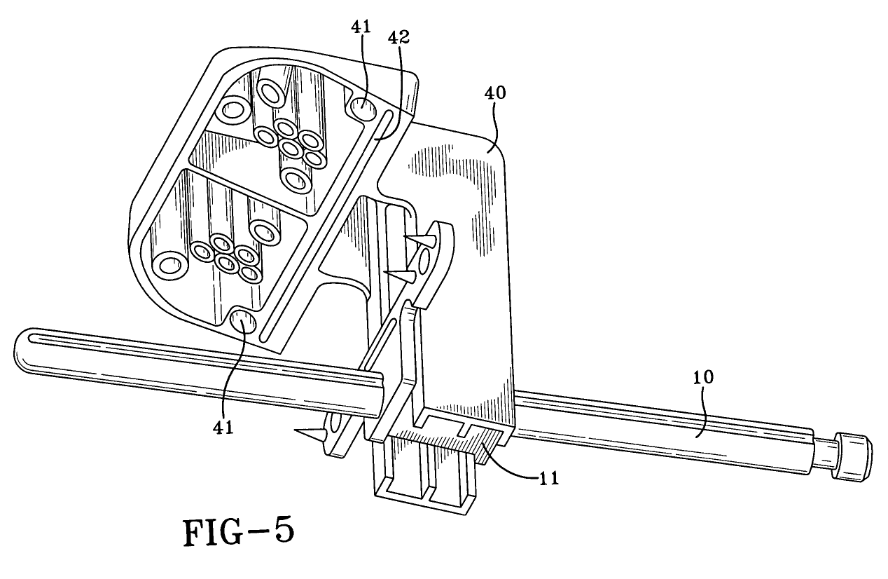

FIG. 5 shows a perspective view of one part of a two part jig embodiment; and

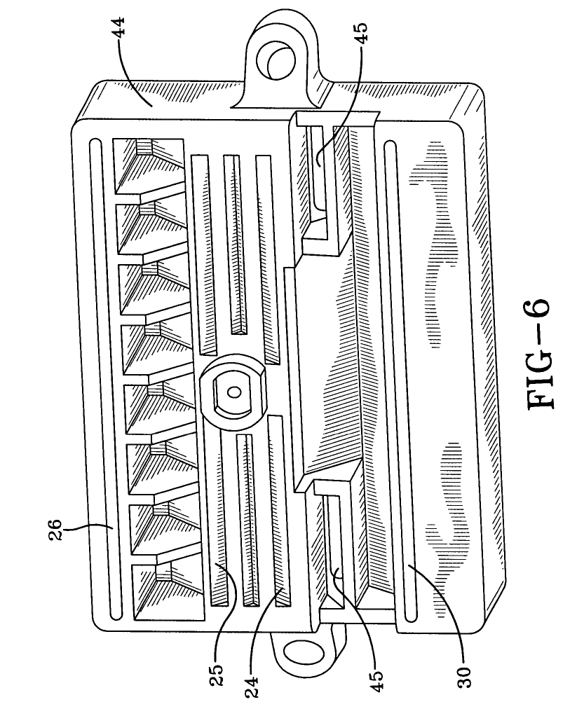

FIG. 6 shows a perspective view of the other part of the two part cutting jig.

The embodiments of the present invention are all made of plastics mouldings and are designed to be supplied as a kit of parts in a sterile pack. Once the surgeon has finished, the kit of parts is disposed with. All the embodiments are designed to explain how the femur is prepared ready to accept an artificial knee joint. It is, of course, necessary to also prepare the tibia but it will be appreciated that a similar procedure will be followed to that which will be described later and also appropriate jigs will be provided which will again be made of plastic mouldings so that they may be disposed of after use.

In preparing for total knee arthroplasty, it is already known that there is a “5 in 1” procedure as well as a “4 in 1” procedure. The first embodiment to be described relates to a 5 in 1 procedure which permits five cuts to be made utilising one cutting jig.

Turning now to FIGS. 1 and 2, the apparatus of the present invention consists of a kit of parts formed by an alignment rod 10 made of metal, a detachable handle (not shown) for attachment to one end of the alignment rod, a rotation guide member, an alignment block 11 arranged to be fitted on to the alignment rod and a cutting jig assembly 12 provided with a stylus 13. The cutting jig assembly 12 is made from a suitable material which can be moulded into a rigid form. Most, if not all the parts may be made from a plastics material but some or parts of some of the kit may be metal. All are disposable.

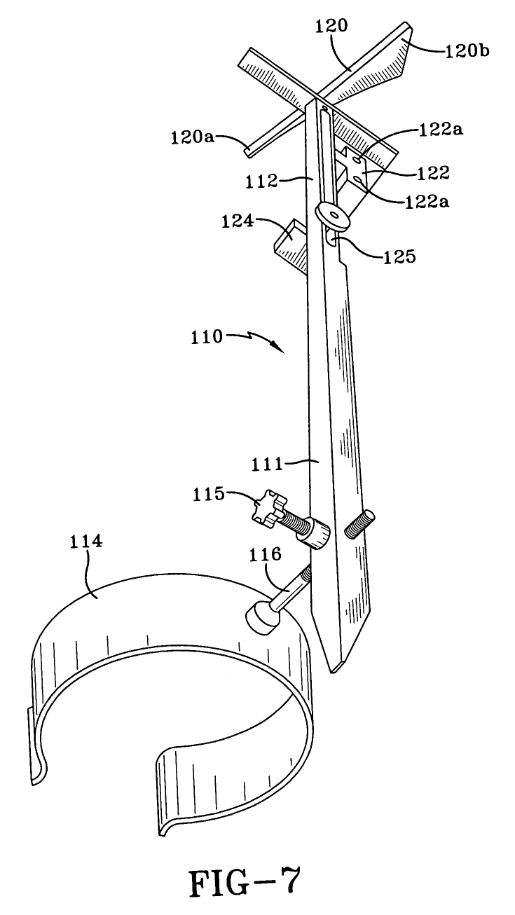

FIG. 7 shows an alignment jig for resectioning the tibia;

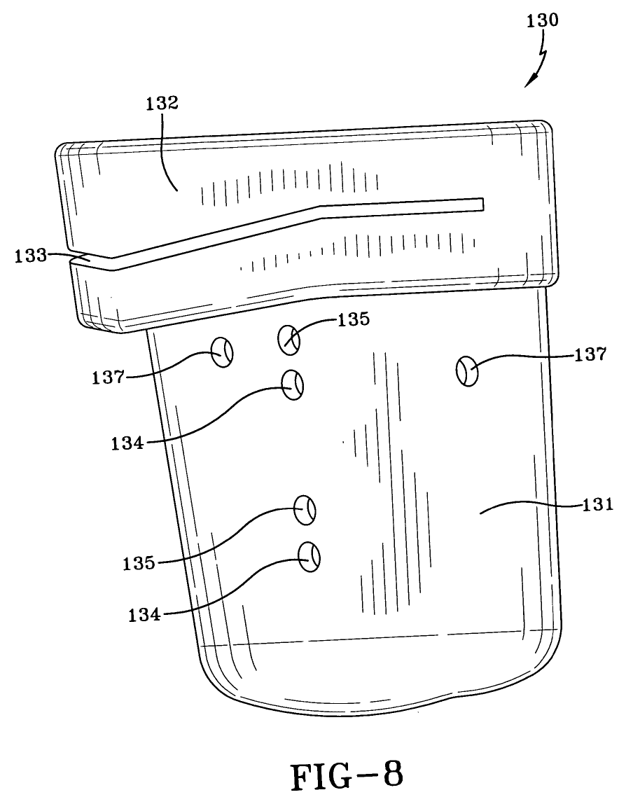

FIG. 8 shows a perspective view of a cutting jig for use in resectioning the tibia;

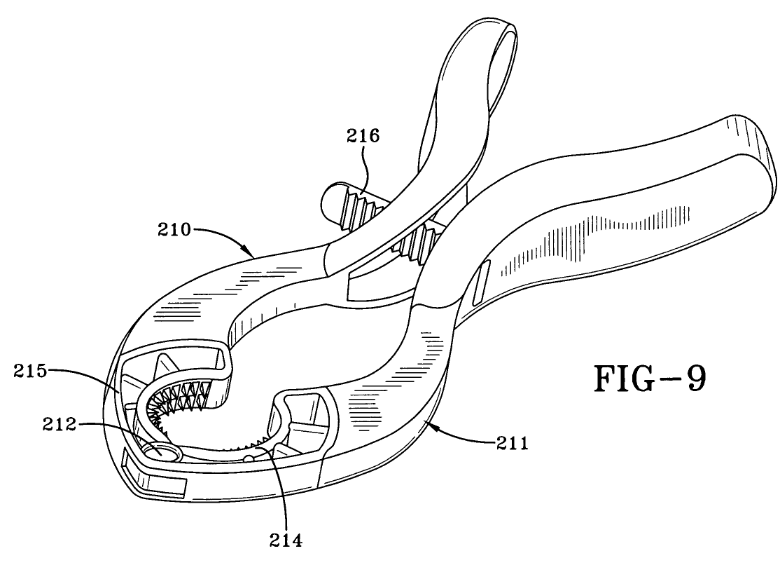

FIG. 9 shows a patella gripper; and

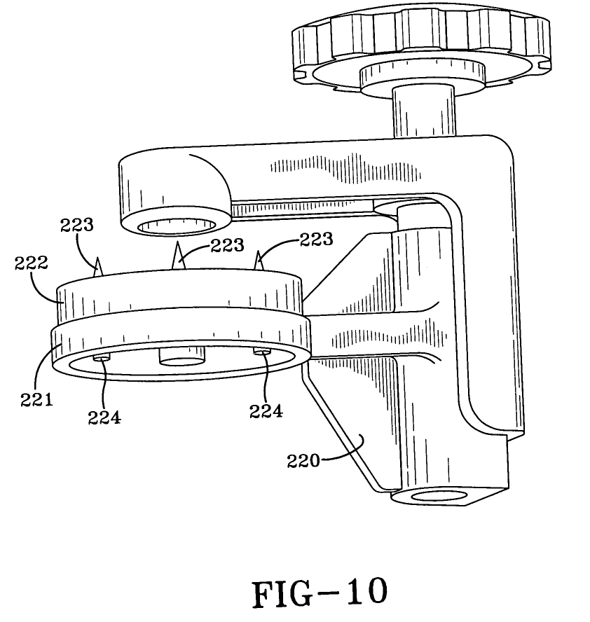

FIG. 10 shows a drill jig.

The cutting jig assembly 12 comprises a main body member 20 which is generally box shaped with a top surface 20a, end surfaces 20b and a sloping front surface 20c at the bottom of which is located the stylus 13. The bottom of the main body member is recessed in order to receive the end of the bone, in this case the femur, which is to be cut to shape.

The top surface 20a is provided with an opening 21 (see FIG. 2), the upper portion of which is generally V-shaped and which extends into the recess in the main member 20. This opening 21 receives a generally V-shaped member 22 which is held in position on the top surface 20a so as to form two angled slots 24 and 25 of a width to permit a tool such as a reciprocating saw to project through the slots for cutting a bone in the recess of the main member.

The top surface 20a is also provided with a slot 26 which extends normal to the top surface 20a and is again of a width to receive a tool for cutting a bone with recess of the main member.

The sloping front surface 20c is also provided with an opening 27 which forms two walls 27a and 27b at right angles to each other as is best seen in FIG. 2. A generally V-shaped member 28 is received in the opening 27 and is held in position on the front surface 20c so as to form two slots 29 and 30 at right angles to each other which extend in the recess in the main member. The slots 29 and 30 are each of a width to receive a tool for cutting a base in the recess.

The slots 24, 25, 26, 29 and 30 are the guides for the five cuts which need to be made to the end of the femur so as to prepare it to receive an artificial knee joint. The main member may be provided with a further slot parallel to slot 29. that can be used as a re-cut if required.

The end surfaces 20b are provided with a number of holes 31 to permit locating pins or screws to be used to fix the assembly 12 in position on the femur.

In use, the surgeon prepares the femur in basically the same manner as before. Initially, a hole is drilled in the centre of the femur making sure that the hole is parallel to the shaft of the femur in both the anteroposterior and lateral projections. The femur is then sized in order to determine the desired alignment block 11 for use with the particular patient, for this purpose a femoral sizing and rotation guide 31 is used to position 2 off headless pins. The guide 31 is removed leaving the headless pins which are then used to set the rotation angle of the subsequent alignment block. A basic design of a rotation guide is shown in FIG. 3.

Alignment blocks 11 each comprise a base member 11a adapted to contact the top of the femur and an alignment member consisting of a column 11b through which the alignment rod 10 is arranged to pass. The column 11b can be at any one of a fixed number of different angles with respect to the base member 11a and the most common angles are 5, 6 and 7. The base member 11a is provided with two slots or holes 11c arranged to receive the two headless pins left in the bone after use of the guide 31, as best seen in FIG. 4.

After selection of the appropriate alignment block 11, the cutting jig assembly 12 is then slipped over the alignment rod and also over the columnar part 11b of the locator block 11. The stylus 13 on the cutting jig assembly is brought into engagement with the femur and locating screws are inserted into holes 31 on either end surface 20b of the cutting jig assembly 12 in order to fix the cutting jig assembly 12 in place on the femur, at this stage the alignment rod is remolded. Once in position, the jig is used by the surgeon in order to determine the precise position of each of the five cuts which are required in order to prepare the femur to accept the artificial joint. The cuts are made in the normal order as indicated by the broken lines 1 to 5 shown in FIG. 2.

After use, all the parts are disposed of.

Turning now to FIG. 4, this also shows a 5 in 1 cutting jig assembly and the same reference numerals are used for the same parts. However, it differs from the jig assembly shown in FIGS. 1 and 2 in that the jig assembly 12 is inserted onto the alignment block 11 by being slid onto the alignment block 11 in a direction normal to the axis of the alignment rod 10 rather than being slipped axially over the alignment rod 10 and alignment block 11. This is achieved by means of a slot 33 in the main body 20 of the assembly 12 and a corresponding slot 34 in the V-shaped member 22. In order to retain the rigidity of the body 20 and the member 22, it is advantageous to form the main body such that the slots 25 and 26 are each constituted by two aligned portions separated by the slots in the recess 20 and 22. In other respects, the construction and technique are similar to that described in relation to the embodiment shown in FIGS. 1 and 2.

Turning now to FIGS. 5 and 6, these show the parts necessary for a 4 in 1 procedure. In this case, the kit instead of having a single cutting jig assembly as was the case in the previous embodiments, is provided with two cutting jigs. The first cutting jig provides for the cutting of the distal femur using a separate cutting guide to that for cutting the remaining cuts for preparing the femur.

As shown in FIG. 5, the alignment rod, sizing and provision of the appropriate alignment block occurs as in the embodiments described above. In this case, however, a cutting block 40 for a single cut is slipped over the columnar portion of the alignment block 11 as in the embodiment described with reference to FIG. 4 and the block 40 is fixed in position by means of screws or pins being inserted through appropriate holes 41 in the cutting block so as to engage the femur. The distal cut can then be made utilising a slot 42 as a guide. Slot 42 may be replicated by another slot, 3 mm offset but parallel. This is only used should a re-cut be necessary. Holes 11c are used to receive 2 off headless pins. Thereafter, the block 40 is removed and a 4 in 1 cutting block 44 as shown in FIG. 6 is attached. Cutting block 44 is positioned on the headless pins using the slots 45 shown (FIG. 6). This determines the anterior/posterior position and also rotation angle. Mediolateral adjustment is achieved by sliding the block along the pin slots. Cutting block 44 provides a jig for cuts 1 to 4 as indicated in FIG. 2.

As with the previous embodiments, the cutting jigs are made of any suitable disposable material.

When preparing the tibia for reception of an artificial knee joint, it is necessary to resection the end of the tibia with a cut perpendicular to the mechanical axis of the leg. It is important that the cut be at the correct height and angle to maintain the mechanical axis of the leg.

The embodiment illustrated in FIGS. 7 and 8 proposes to provide separate alignment and cutting jigs. Firstly, the alignment jig will be described in relation to FIG. 7. The alignment jig consists principally of a main member generally indicated by the reference numeral 10 which comprises a first portion 111 which telescopically receives a second portion 112 so as to enable the length of the main member 110 to be altered to suit the size of the tibia to be resectioned.

Near one end of the portion 111, is fixed a leg engaging member in the form of a clip 114 which is arranged to hold in position the bottom of the main portion 111 of the jig near the patient's ankle. An adjustment means in the form of a grub screw arrangement 15 operating on a stem 116 projecting from the clip 114 permits the main portion 111 to be moved forwards and away from the clip so as to ensure that the main portion 111 lies parallel to the tibia.

The telescopic portion 112 of the main member is provided with a stylus 120 that extends transverse to the axis of the portion 112 and is arranged to contact the end of the tibia to be resectioned. The stylus 120 is elongate and the ends are provided with two different sized projections 120a, 120b depending on whether the surgeon wishes to resection based on the highest point of the end of the femur or the lowest point on the femur. Consequently, the stylus 120 is rotatably mounted in the end of the portion 112.

The portion 112 is also provided with a location plate 122 having two through holes 122a which enable two holes to be drilled in the tibia to permit two threaded, headless pins to be accurately placed in the tibia for the reception and accurate location of the tibia cutting jig. The plate 122 is shown as being positioned to one side of the portion 112 which means that the headless locating pins will be off axis. This is because the cutting jigs are either left or right handed to cause the resectioning cut to be at the correct angle.

The alignment jig is formed with a separate, adjustable spacer plate 124 so as to ensure that the jig will be set at the correct distance from the tibia. It is rendered adjustable in view of the fact that it is moveable in a slot 125 in the portion 112 which permits the correct placement of the plate 124 against the tibia but still permits the overall length adjustment to be achieved.

It is to be noted that relative rotation between the two sections 111 and 112 of the main member 110 is prevented. In this case this is achieved by utilising rectangular sectioned members but could be achieved in any other convenient fashion as desired.

In operation, the alignment jig is manipulated in much the same manner as is the conventional alignment tool. However, once the location plate 122 is accurately placed in position, two holes are drilled into the tibia and the two threaded headless pins referred to above are placed in the holes.

Referring now to FIG. 8, this shows a tibia cutting jig 130 which comprises a mounting portion 131 and a head portion 132 formed with a slot 133 which forms a guide for a cutting saw.

It is to be noted that the cutting jig is either a left or right handed jig but is always medially biased. The jig is made of a single piece of moulded construction and if not made of metal may be made of plastic in which case the slot should be lined with a metal insert.

The mounting portion of the cutting jig 130 is provided with a pair of holes 134 so that the cutting jig can be mounted on the headless locating pins that have previously been inserted into the tibia. One or more further pairs of holes such as those indicated by the reference numeral 135 can be provided to allow for repositioning of the height of the jig. In any event, once the jig is in position on the locating pins, it is fixed in position by driving two fixing pins into the tibia through the fixing holes 137.

Once securely located on the tibia, cutting can be commenced and it will be noted that the lateral end of the slot is open to allow for optimum saw blade movement.

After use, the cutting jig can be discarded while the alignment jig can be autoclaved as before.

The two jigs of FIGS. 7 and 8 will normally form a kit of parts together with the instruments described for resectioning the femur described in our earlier application.

Once the tibia has been cut and finished, the tibia can be prepared in the conventional manner depending on whether or not the tibial fixation plate is provided with a stem.

Referring now to FIGS. 9 and 10, kit of instruments for operating on the patella comprises a gripper and a drill jig. As shown in FIG. 9, the patella gripper comprises a tool arranged so as to provide a continual compressive force on the periphery of the patella and is provided with handles to enable the surgeon to hold or manipulate the patella at will.

The arrangement shown in the drawing is such that the tool is made of two plastics parts 210,211 which are hinged together at one end 212. Each of the parts 210,211 adjacent the end 212 is either formed with or faced with metal. An interior surface 214 of each metal part is arcuately formed and provided with serrations while the upper and lower metal surfaces 215 are arranged to form a saw guide.

The two parts 210,211 are provided with an interlocking arrangement such as that shown by the ratchet 216. In use, the gripper is opened to a degree sufficient to allow the patella to be received between the serrated surfaces 214 and then the parts 210 and 211 are squeezed together until the patella is securely gripped. The parts 210,211 are then held in this position due to the ratchet 216. If desired, the plastic parts 210,211 can be arranged so that the handles will deform if too much pressure is applied to the patella.

Once gripped, the patella can be adjusted within the jaws 214 so as to expose a desired amount of bone for resectioning. A reciprocating saw is then sed to resection the specified portion of the patella utilising the surfaces 215 of the clamp as a saw guide.

Once the surface has been resectioned, a drill jig such as that shown in FIG. 10 is used.

The drill jig comprises a main body portion 220 provided with a jig locating portion 221 and a patella jig portion 222 which is made of metal and provided with sharp projections 223. The patella is arranged to be received between the jig portion 222 and a clamping member 223. Once the patella is securely held in position on the sharp projections 223, holes can be drilled through guide holes 224 and 222 and portion 221 into the patella. These holes form the location holes for a surfacing member which can then be applied to the patella.

It will be seen that the advantage of the embodiment of the invention, shown in FIGS. 9 and 10 is that the patella gripper applies continues pressure to the periphery of the patella to retain it within the gripper without the surgeon himself applying any pressure. Further, all or substantially all of the parts can be made of plastics material and so the instruments can be disposed of, if desired, while those parts likely to come into contact with a saw or drill are made with or faced with metal.

1. A cutting jig for use in resectioning an end of a bone during orthopaedic surgery, comprising a generally parallel epipedal member (12) having a top face (20a), a bottom face and four side faces (20a,20b) with the bottom face being recessed in order to receive the end of the bone to be resectioned, a plurality of slots (24,25,27,29,30) through the member (12) for receiving a cutting implement, a generally medial opening (33) extending from the bottom face for receiving an alignment member and having a shaped cross section whereby to mate with the alignment member in a predetermined orientation, and a plurality of fixing elements (31) in at least two side walls arranged to enable fixing the cutting jig to the bone.

2. A cutting jig according to claim 1 wherein the shape of a cross section of the opening is rectangular.

3. A cutting jig according to claim 1, wherein the generally epipedal member is a made of moldable material.

4. A cutting jig according to claim 3, wherein the moldable material is plastics.

5. A cutting jig according to claim 4 wherein the plastics material in transparent.

6. A cutting jig according to claim 4 wherein the slots (24,25,27,29,30) are lined with metal.

7. A cutting jig according to claim 6 wherein the metal is stainless steel.

8. A cutting jig according to claim 1 wherein there are four slots.

9. A cutting jig according to claim 1 wherein there are five slots.

10. A kit of parts for use in resectioning an end of a bone during orthopaedic surgery, comprising a cutting jig (12) and a alignment tool (10,11), the cutting jig comprising a generally parallel pipedal member having a top face (20a) a bottom face and four side faces (20b,20c), the bottom face being recessed in order to receive the end of the bone to be resectioned, a plurality of slots (24,25,27,29,30) through the member (12) for receiving a cutting implement, a generally medial opening (33) extending from the bottom face for receiving the alignment tool (10,11) and having a shaped cross section whereby to mate with the alignment tool (10,11) in a predetermined orientation, and a plurality of fixing elements (31) in at least two side walls (20b) arranged to enable fixing the cutting jig (12) to the bone.

11. A kit of parts according to claim 10, wherein the alignment tool comprises an elongate metal rod (10) and a separate alignment member (11) arranged to have a bore for receiving the rod.

12. A kit of parts according to claim 11, wherein the alignment member (I 1) comprises a main portion (11b) provided with a bore for receiving the rod, and a flange (11a) provided on the main member and extending in opposite directions therefrom to facilitate fixing of the alignment member to the end of the bone to be resectioned.

13. A kit of parts according to claim 11, wherein the alignment member (11) comprises a main portion (11b) having a top, bottom and side walls, the bore extending through the member from the top to the bottom at an angle to the direction at right angles to the bottom wall.

14. A kit of parts according to claim 13, wherein the bottom wall of the main portion (11b) is provided with a flange (11a) extending in opposite directions therefrom to facilitate fixing the alignment member to the end of bone to be resectioned.

15. An alignment tool for use with a cutting jig in a kit of parts for resectioning the end of a bone during orthopaedic surgery: the tool comprising an elongate rod (10) and separate alignment block (11) having a top, bottom and side walls with means defining a bore through the block from the top wall to the bottom wall for receiving the separate alignment rod (10), the bore being at an angle to the direction at right angles to the bottom wall.

16. An alignment tool according to claim 15 wherein the bottom wall of the block (11b) is provided with a flange (11a) which extends from the block in opposite directions and is provided with projections for engagement with the end of the bone to resectioned in order to position the block on the bone.

17. An alignment tool according to claim 15 wherein the alignment block (11b) is of generally rectangular cross-section with four side walls.

18. A kit of parts for use in resectioning an end of bone during orthopaedic surgery comprising an alignment tool (10,11) and a cutting jig (12) having a plurality of slots (24,25,27,29,30) for a cutting tool, the alignment tool comprising an elongate rod (10) and an alignment block (11) having a top, bottom and side walls with means defining a bore through the block from the top wall to the bottom wall for receiving the alignment rod (10), the bore being at an angle to the direction at right angles to the bottom wall.

19. A kit of parts according to claim 18 wherein the bottom wall of the block (11b) is provided with a flange (11a) which extends from the block in opposite directions and is provided with projections for engagement with the end of the bone to be resectioned in order to position the block on the bone.

20. A kit of parts according to claim 19 wherein the cutting jig (12) has an opening (33) for receiving the alignment block (11b), which opening has a shape which mates with a corresponding shape of the alignment block whereby the jig adopts a predetermined orientation with respect to the alignment block when the block is in the bore.

21. A kit of parts according to claim 20 wherein the opening in the cutting jig is at least partly of rectangular cross-section.

22. A kit of parts according to claim 18 wherein the cutting jig has four slots for receiving a cutting tool.

23. A kit of parts according to claim 18 wherein the cutting jig has five slots for receiving a cutting tool.

24. A method for resectioning an end of a bone during orthopaedic surgery using a kit of parts comprising an alignment tool having an elongate alignment rod and separate alignment block having a bore for receiving the rod, and cutting jig having a plurality of slots for receiving a cutting tool and an opening for receiving the alignment block, the method comprising
exposing the end of the bone to be resectioned:
inserting the alignment rod axially into the exposed end of the bone:
selecting an appropriate alignment block:
sliding the selected alignment block on to the alignment rod using the bore in the block:
attaching the alignment block to the end of the bone:
placing the cutting jig on the alignment tool with the alignment block received in the opening in the cutting jig:
fixing the cutting jig to the bone to be resectioned:
removing the alignment rod:
sequentially inserting a cutting tool into the slots in the cutting jig whereby to resection the end of the bone.

25. A kit of parts for use in resectioning an end of a bone during orthopaedic surgery comprising an alignment jig (110) and a cutting jig (130), the alignment jig including means (122) for positioning pins at a preselected location on the bone to be resection, and the cutting jig (130) being a block of molded material having apertures (134) for receiving the positioning pins, further apertures (137) for receiving means for fixing the cutting jig to the bone, and at least one slot (133) for receiving a cutting tool for resectioning the end of the bone.

26. A kit of parts according to claim 25, wherein the cutting jig is made of plastics material.

27. A kit of parts according to claim 26, wherein the slot (133) in the cutting jig is lined with metal.

28. A kit of parts according to claim 25, wherein there are two cutting jigs each with a slot of opposite hand and medially biased.

29. A kit of parts according to claim 25, wherein the alignment jig (110) comprises first and second elongate members (111,112) which are telescopically connected together whereby the length of the jig is adjustable.

30. A kit of parts according to claim 29 wherein the first member (111) is provided with a resilient clip (114) for attaching one end of the alignment jig (110) to a body part.

31. A kit of parts according to claim 30, wherein the resilient clip (114) is spaced from said first member (111).

32. A kit of parts according to claim 31, comprising adjustable means (115,116) for changing the spacing between the resilient clip (114) and said first member.

33. A kit of parts according to claim 29, wherein the second member (112) is provided with a spacer member (124).

34. A kit of parts according to claim 33, wherein the second member (112) is provided with an elongate slot (125) extending along the length of the second member, and the spacer member (124) is mounted for movement in the slot whereby the position of the spacer member is changeable.

35. A kit of parts according to claim 29, wherein the positioning means (122) is a block having a plurality of apertures (122a), the block being located on one side of the second member and a predetermined distance from a free end of the second member.

36. A kit of parts according to claim 35, wherein the free end of the second member is provided with a stylus (120) for contacting the end of the bone.

37. A kit of parts according to claim 36, wherein the stylus (120) is a elongate member pivotally mounted to the free end of the second member, the ends of the elongate member being provided with projections (120a,120b) of different lengths.

38. A cutting jig for use in resectioning an end of a bone during orthopaedic surgery, comprising a unitary block (130) of molded material having a first plurality of apertures (134) for receiving positioning pins fixed to the bone to be resectioned, a further plurality of apertures (137) for receiving means for fixing the cutting jig to the bone, and a slot (133) for receiving a cutting tool.

39. A cutting jig according to claim 38, wherein the jig has a main body portion (132) arranged to extend round a part of the circumference of the end of the bone and a secondary portion (131) extending from the main body portion and along a portion of the length of the bone, the main portion (132) being provided with the lot (133) and the secondary portion (131) being provided with the apertures 134,137) for receiving positioning pins and for fixing the jig to the bone.

40. A cutting jig according to claim 38, wherein the slot (133) has one end which is open to allow for optimum movement of a cutting tool.

41. A cutting jig according to claim 38, wherein the first plurality of apertures includes two sets of apertures (134,135) at different locations whereby the cutting jig may be fixed to the bone at two different positions.

42. An alignment jig for enabling the accurate location of positioning pins on a bone, comprising:
first and second elongate members (111,112) which are telescopically connected together whereby the length of the jig is adjustable, the first member (111) being provided with resilient clip (114) for attaching one end of the jig to a body part, and the second member (112) being provided with a spacer member (124) for maintaining the elongate members spaced from the bone and with means (122) for positioning pins at a preselected location on the bone.

43. An alignment jig according to claim 42, wherein at least one of the resilient clip and the spacer member (124) is adjustable whereby to maintain the jig (110) parallel to and at the correct distance from the bone.

44. An alignment jig according to claim 43, wherein the second member (112) is provided with an elongate slot (125) extending along the length of the second member, and the spacer member (124) is mounted for movement in the slot to a desired position.

45. An alignment jig according to claim 44, wherein the positioning means (122) is in the form of a block having a plurality of apertures (122a), the block being located on one side of the second member (112) and a predetermined distance from a free end of the second member.

46. An alignment jig according to claim 45, wherein the free end of the second member (112) is provided with a stylus (120) for contacting the end of the bone.

47. An alignment jig according to claim 46, where the stylus (120) is a elongate member pivotally mounted to the free end of the second member, the ends of the elongate member being provided with projections (120a, 120b) of different lengths.

48. A kit of parts for use in resectioning the tibia and femur during orthopaedic surgery, comprising first and second kits, each for a respective bone, the first kit comprising a cutting jig (12) having a top face (20a), a bottom face and four side faces (20a,20b) with the bottom face being recessed in order to receive the end of the bone to be resectioned, a plurality of slots (24,25,27,29,30) through the member (12) for receiving a cutting implement, a generally medial opening (33) extending from the bottom face for receiving an alignment member and having a shaped cross section whereby to mate with the alignment member in a predetermined orientation, and a plurality of fixing elements (31) in at least two side walls arranged to enable fixing the cutting jig to the bone, and the second kit comprising a further alignment jig (110) and a cutting jib (130), the alignment jig including means (122) for positioning pins at a preselected location on the bone to be resection, and the cutting jig (130) being a block of molded material having apertures (134) for receiving the positioning pins, further apertures (137) for receiving means for fixing the cutting jig to the bone, and at least one slot (133) for receiving a cutting tool for resectioning the end of the bone.

49. A gripping device for gripping at least a portion of the perimeter of an article comprising two levers (210,211) pivotally connected to each other at one end (212) with each lever having two sections, one section forming a manually operable part and the other section forming a shaped gripping part (214,215), wherein the shaped gripping part has a material which is harder than the material forming the manually operable part.

50. A device according to claim 49, wherein the harder material is a surface provided on a base member of the same material as that forming the manually operable part.

51. A device according to claim 49, wherein the material forming the manually operable part is a plastics material and the harder material is metal.

52. A device according to claim 49 wherein the metal is stainless steel.

53. A device according to claim 49, wherein the two levers are additionally connected together by a ratchet mechanism (216).

54. A kit of parts for use in resurfacing the patella, comprising a gripping device (210,211) and a drilling jig (220,221,222), the gripping device comprising two levers (210,211) pivotally connected to each other at one end (212) with each lever having two sections, one section forming a manually operable part and the other section forming a shaped gripping part (214,215), and the drilling jig comprises a main body portion (220) provided with a jig locating portion (221) and a jig body (222), the jig body (222) being made of a material different to that of the main body and jig locating portion.

55. A kit of parts according to claim 54, wherein the jig body (222) is made of metal and the other parts (220,221) of the drilling jig are made of plastics material.

56. A kit of parts comprising the kit according to claim 48 and the kit according to claim 54.
